(19) United States
(12) Patent Application Publication
(10) Pub. No.: US 2021/0324391 A1
(43) Pub. Date: Oct. 21, 2021

(54) RECOMBINANT MICROORGANISM, PREPARATION METHOD THEREFOR AND APPLICATION THEREOF IN PRODUCING COENZYME Q10

(71) Applicants: ZHEJIANG NHU COMPANY LTD., Zhejiang (CN); HEILONGJIANG NHU BIOTECHNOLOGY COMPANY TLD., Hei Longjiang (CN); SHANGYU NHU BIOLOGICAL CHEMICAL CO., LTD., Zhejiang (CN); ZHEJIANG UNIVERSITY, Zhejiang (CN)

(72) Inventors: Hongwei Yu, Zhejiang (CN); Shenfeng YUAN, Zhejiang (CN); Yongqiang ZHU, Zhejiang (CN); Kai YU, Zhejiang (CN); Zhirong CHEN, Zhejiang (CN); Yong LI, Hei Longjiang (CN); Guisheng QIU, Zhejiang (CN); Xiaoqing LIU, Zhejiang (CN)

(21) Appl. No.: 17/271,356

(22) PCT Filed: May 31, 2019
(86) PCT No.: PCT/CN2019/089437

§ 371 Date: Feb. 25, 2021

(30) Foreign Application Priority Data
Aug. 28, 2018 (CN) 201810984954.9

(51) Int. Cl. C12N 15/74; C07K 14/245; C12P 7/66; C12N 1/20

(52) U.S. Cl. CPC C12N 15/74; C12N 1/205; C12P 7/66; C07K 14/245

(57) ABSTRACT

A recombinant microorganism, a preparation method thereof and its application in the production of coenzyme Q10. Specifically, the present disclosure provides a method of exogenously introducing a gene encoding the global regulatory protein irrE to construct a recombinant microorganism. This recombinant microorganism is suitable for producing coenzyme Q10 by fermentation method, and is particularly suitable for producing oxidized coenzyme Q10. The recombinant microorganism of the present disclosure has stress resistance, and has good tolerance against harsh environments including high osmotic pressure and high redox potential, thus making it possible to significantly increase the yield of coenzyme Q10, especially the yield of oxidized coenzyme Q10.

The present application is a national stage entry from PCT/CN2019/089437, filed May 31, 2019. Application PCT/CN2019/089437 claims priority from Application CN201810984954.9 filed on Aug. 28, 2018 in China. The entire contents of these applications are incorporated herein by reference in their entirety. In the present disclosure relates to the field of biotechnology, specifically relates to a recombinant microorganism and its application in the production of coenzyme Q10.

### REFERENCE TO SEQUENCE LISTING SUBMITTED VIA EFS-WEB

This application includes an electronically submitted sequence listing in .txt format. The .txt file contains a sequence listing entitled “LLIU-103_A_ST25.txt” created on Apr. 13, 2021 and is 3,489 bytes in size. The sequence listing contained in this .txt file is part of the specification and is hereby incorporated by reference herein in its entirety.

### BACKGROUND

Coenzyme Q10 (CoQ10) is also known as ubiquinone or decenequinone, and its chemical name is 2, 3-dimethoxy-5-methyl-6-decaprenylbenzoquinone. The biological activity of coenzyme Q10 comes from the redox property of its quinone ring and the physical and chemical properties of its side chain. It is a natural antioxidant and cell metabolic activator produced by cell itself, and has functions such as anti-oxidation function, eliminating free radicals, improving the body immunity and anti-aging function. Coenzyme Q10 is widely used in the clinical treatment of diseases such as various types of heart disease, cancer, diabetes, acute and chronic hepatitis and Parkinson's disease, and also has many applications in foods, cosmetics and anti-aging health care products.

Currently, microbial fermentation method is the main production method of coenzyme Q10. Using the microbial fermentation method to produce coenzyme Q10 has great competitive advantages in terms of both quality and safety of the products, and is suitable for large-scale industrial production. However, external pressures from various harsh environments are often encountered during the growth or proliferation process of microorganism, especially in an industrial fermentation environment. For example, there is certain fluctuation in conditions such as the osmotic pressure, pH, dissolved oxygen and nutrient substances of the fermentation environment. The growth of microorganism is affected by said fluctuation and has the characteristic of not being easy to control, and the production of coenzyme Q10 is also unstable. Meanwhile, it is difficult to further increase the biomass in the industrial production due to the limitation of the fermentation environment. Therefore, it is necessary to enhance the tolerance of the coenzyme Q10-producing strain against harsh environments, so as to further increase the yield of coenzyme Q10.

The reported research on the related technology of the fermentation of coenzyme Q10 mainly focuses on improving the production level of coenzyme Q10 by transformation via genetic engineering technology, or investigating the influence on the synthesis of products by the mutagenesis treatment of strain and experiments in which single factor is optimized and adjusted. Also, some patent documents have reported that the on-line control of key parameters in the fermentation process is adopted to optimize the fermentation process. Although this research and technology have certain functions, none of them fundamentally enhances the tolerance of the coenzyme Q10-producing strain against harsh environments and thus achieving the purpose of increasing the production capacity.

Thus, it would be desirable to provide a method of generating a recombinant microorganism that addresses these and other concerns.

### SUMMARY

Disclosed is a method for generating a recombinant microorganism that includes the steps of cloning a gene encoding a global regulatory protein irrE from a parent strain comprising the gene encoding the global regulatory protein irrE; ligating the gene encoding the global regulatory protein irrE to a vector, and constructing a recombinant vector comprising the gene encoding the global regulatory protein irrE; and introducing the recombinant vector into a host cell so as to obtain the recombinant microorganism and a recombinant microorganism resulting therefrom.

### BRIEF DESCRIPTION OF THE DRAWINGS

The various features, advantages and other uses of the present apparatus will become more apparent by referring to the following detailed description and drawing in which:

FIG. 1 is a map of the original plasmid pBBR1MCS-2;

FIG. 2 is a map of the recombinant plasmid pBBR1MCS-2-G-proPB-IrrE;

FIG. 3 is an electrophoretogram of the recombinant microorganism RSP-CE which comprises the gene encoding the global regulatory protein irrE but has not been subjected to osmoregulated promoter proPB replacement; and

FIG. 4 is an electrophoretogram of the recombinant microorganism RSP-BE which comprises the gene encoding the global regulatory protein irrE and has been subjected to osmoregulated promoter proPB replacement.

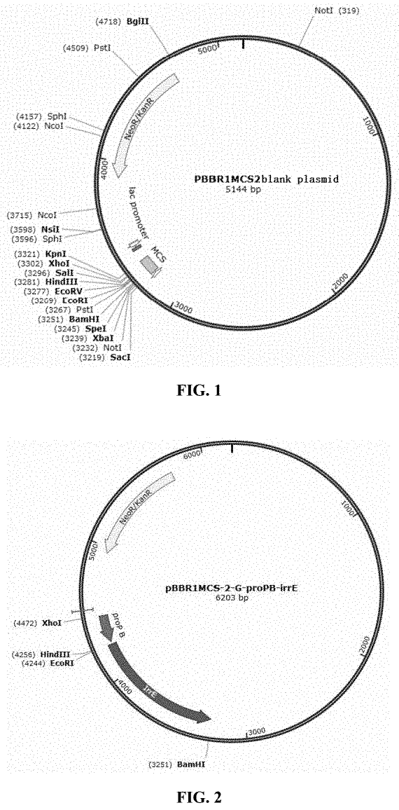

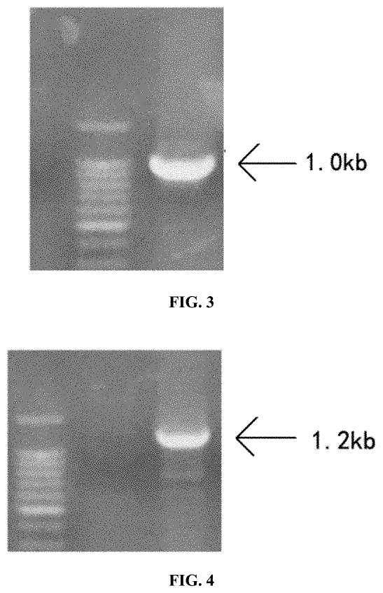

### DETAILED DESCRIPTION

Problems exist in various processes. The process disclosed in CN 105420417A is directed to synergistical control of the fermentation process of coenzyme Q10 by adjusting the oxygen consumption rate (dissolved oxygen) and the electrical conductivity (feeding rate of nutrients). CN 104561154A discusses adjusting the technological parameters using the shape of the microbe during the fermentation process as the basis for judgment. CN 103509729B improves the ability of a microorganism to synthesize coenzyme Q10 by transforming Rhodobacter sphaeroides. A common feature of these technologies is that the produced coenzyme Q10 is a mixture of oxidized coenzyme Q10 and reduced coenzyme Q10, and the proportion of reduced coenzyme Q10 is relatively high. In particularly, in the technology disclosed in U.S. Pat. No. 7,910,340 B2, the content of reduced coenzyme Q10 in coenzyme Q10 produced by the microorganism is 70% or more after the completion of fermentation.

CN 108048496A discloses a fermentation production method of oxidized coenzyme Q10. The technology disclosed therein enables the strain to produce oxidized coenzyme Q10 with a high content by regulating the oxidation-reduction potential (ORP) at the later stage of the synthesis and accumulation stage of coenzyme Q10, in which the content of oxidized coenzyme Q10 is 96% or more. However, this method does not solve the problems such as the accumulation of metabolites in the microbe resulted by the oxidative stress on the producing strain caused by high redox potential, and the inhibition of microbe growth.

The method as disclosed herein is predicated on one or more unexpected discoveries such as those solving problems such as the accumulation of metabolites in the microbe resulted by the oxidative stress on the producing strain caused by high redox potential, and the inhibition of microbe growth.

### Problems to be Solved by the Disclosure

In order to solve the above-mentioned problems existing in the fermentation process of coenzyme Q10, especially in the fermentation process of oxidized coenzyme Q10, and in order to enhance the tolerance of the coenzyme Q10-producing strain against harsh environments, the present disclosure constructs a recombinant microorganism. The gene encoding the global regulatory protein irrE is introduced exogenously, thereby enhancing the tolerance of the coenzyme Q10-producing strain against harsh environments. It is suitable for the production of coenzyme Q10 by fermentation method, especially suitable for the production of oxidized coenzyme Q10.

Said recombinant microorganism has stress resistance, and has good tolerance to harsh environments including high osmotic pressure and high redox potential. These properties of the microorganism in the fermentation process of coenzyme Q10 may be well improved by over-expressing the gene encoding the global regulatory protein irrE as set forth in SEQ ID NO: 1.

The global regulatory protein irrE, as a switch that activates the related important genes in the organism, plays a central regulatory role in the pathways of the repair of DNA damage and the protection from the response of radiation stress. Introducing the gene encoding the global regulatory protein irrE exogenously, on one hand, may enhance the tolerance of the microorganism against a variety of stresses such as osmotic pressure, oxidation, radiation and heat. On the one hand, the logarithmic growth phase of the strain is prolonged and further accumulation of biomass is promoted, on the other hand, the strain is allowed to keep vigorous growth and metabolic activity during the fermentation process, so as to increase the yield of coenzyme Q10, especially the yield of oxidized coenzyme Q1.

Preferably, the present disclosure may also knock out a promoter in the recombinant vector in which said promoter controls the expression of the gene encoding the global regulatory protein irrE, and then insert other different promoter(s) by promoter replacement, so as to further regulate the expression of the gene encoding the global regulatory protein irrE.

The inserted promoter is preferably an osmoregulated promoter proPB set forth in SEQ ID NO: 2. The initial expression intensity of this promoter is low, but its expression intensity will increase with the increase of osmotic pressure, thus enabling the expression amount of the global regulatory protein irrE to increase with the increase of osmotic pressure, and enhancing the tolerance of the microorganism to stresses of different intensities.

### Solution to the Problems

The present disclosure provides a method for generating a recombinant microorganism, said method comprises the following steps:
step a. cloning a gene encoding a global regulatory protein irrE from a parent strain comprising the gene encoding the global regulatory protein irrE;
step b. ligating the gene encoding the global regulatory protein irrE to a vector, and constructing a recombinant vector comprising the gene encoding the global regulatory protein irrE; and
step c. introducing the recombinant vector into a host cell so as to obtain the recombinant microorganism.

In the above-mentioned method of the present disclosure, said step b includes knocking out a promoter in the recombinant vector in which said promoter controls the expression of the gene encoding the global regulatory protein irrE and then inserting other different promoter(s) by promoter replacement, so as to further regulate the expression of the gene encoding the global regulatory protein irrE.

In the above-mentioned method of the present disclosure, the promoter inserted in said step b is an inducible promoter, preferably an osmoregulated promoter proPB. Said osmoregulated promoter proPB is obtained from a polynucleotide molecule or a polynucleotide sequence comprising a partial nucleotide sequence of at least 70 consecutive nucleotides of SEQ ID NO: 2, preferably comprising at least 100 consecutive nucleotides of SEQ ID NO: 2, more preferably comprising at least 150 consecutive nucleotides of SEQ ID NO: 2, and most preferably comprising the whole nucleotide sequence of SEQ ID NO: 2. Said polynucleotide sequence has at least 60% homology, preferably at least 80% homology and more preferably at least 90% homology with SEQ ID NO: 2. Preferably, said osmoregulated promoter proPB is a nucleotide sequence set forth in SEQ ID NO: 2. Said osmoregulated promoter proPB is isolated from a bacterium which is preferably Escherichia and more preferably Escherichia coli.

In the above-mentioned method of the present disclosure, the vector in said step b is selected from pBR322 and its derivatives, pACYC177, pACYC184 and its derivatives, RK2, pBBR1MCS-2 and a cosmid vector and its derivatives, and is preferably pBBR1MCS-2.

In the above-mentioned method of the present disclosure, said step a includes designing a primer according to a DNA sequence set forth in SEQ ID NO: 1, using a genomic DNA extracted from the parent strain as a template, and synthesizing the gene encoding the global regulatory protein irrE by a PCR method.

In the above-mentioned method of the present disclosure, the gene encoding the global regulatory protein irrE in said step a is obtained from a polynucleotide molecule or a polynucleotide sequence comprising a partial nucleotide sequence of at least 100 consecutive nucleotides of SEQ ID NO: 1, preferably comprising at least 300 consecutive nucleotides of SEQ ID NO: 1, more preferably comprising at least 600 consecutive nucleotides of SEQ ID NO: 1, and most preferably comprising the whole nucleotide sequence of SEQ ID NO: 1. Said polynucleotide sequence has at least 60% homology, preferably at least 80% homology and more preferably at least 90% homology with SEQ ID NO: 1. Preferably, said gene encoding the global regulatory protein irrE is a nucleotide sequence represented by SEQ ID NO: 1. Said parent strain is a bacterium, preferably Deinococcus, more preferably selected from the group consisting of Deinococcus radiodurans, Deinococcus deserti, Deinococcus gobiensis and Deinococcus proteolyticus, and most preferably Deinococcus radiodurans.

In the above-mentioned method of the present disclosure, the way of introducing in said step c is selected from transformation, transduction, conjugative transfer and electroporation. Said host cell is selected from bacteria or fungi, preferably a bacterium of Rhodobacter, and more preferably Rhodobacter sphaeroides. Preferably, said step c includes transforming the recombinant vector obtained in said step b to an Escherichia coli S17-1 competent cell and then introducing the recombinant vector into the host cell by conjugative transfer, so as to obtain a genetically stable recombinant microorganism.

The present disclosure also provides a recombinant microorganism, which at least comprises the above-mentioned gene encoding the global regulatory protein irrE and the osmoregulated promoter proPB.

The present disclosure also provides a method for producing coenzyme Q10, which comprises generating a recombinant microorganism by using the above-mentioned method and producing coenzyme Q10 by using the above-mentioned recombinant microorganism.

The present disclosure also provides a method for producing oxidized coenzyme Q10, which comprises generating a recombinant microorganism by using the above-mentioned method and producing oxidized coenzyme Q10 by using the above-mentioned recombinant microorganism.

### Effects of the Present Disclosure

After intensive research conducted by the inventors, it has been surprisingly found that a recombinant microorganism constructed by the introduction of the gene encoding the global regulatory protein irrE and the promoter replacement, especially a recombinant Rhodobacter sphaeroides has stress resistance, and has good tolerance against harsh environments including high osmotic pressure and high redox potential. On the one hand, the logarithmic growth phase of the strain is prolonged and further accumulation of biomass is promoted, on the other hand, the strain is allowed to keep vigorous growth and metabolic activity during the fermentation process.

In addition, in the existing direct production technologies of oxidized coenzyme Q10, a large amount of oxidized coenzyme Q10 is accumulated in the cell at the later stage of fermentation, and the microbe itself is subjected to strong oxidative stress. The recombinant microorganism of the present disclosure enhances the tolerance of microbe against oxidative stress and significantly increases the potency of oxidized coenzyme Q10, thus contributing to increasing the proportion of oxidized coenzyme Q10 in the total amount of coenzyme Q10.

In addition, during the construction of the above-mentioned recombinant microorganism in the present disclosure, it has also been found that after replacing the promoter that controls the expression of the gene encoding the global regulatory protein irrE with proPB promoter, it is possible to regulate the expression of the global regulatory protein irrE by changing the osmotic pressure, to enhance the stress resistance of the coenzyme Q10-producing strain by stages, and to satisfy the needs of microbe tolerance against harsh environments at different stages of the fermentation process, thereby facilitating the production of coenzyme Q10, especially the production of oxidized coenzyme Q10.

1. Microorganism Used to Produce Coenzyme Q10

The present disclosure constructs a recombinant microorganism by exogenously introducing the gene encoding the global regulatory protein irrE, so as to enhance the tolerance of the coenzyme Q10-producing strain against harsh environments. It is suitable for the production of coenzyme Q10 by fermentation method, and is particularly suitable for the production of oxidized coenzyme Q10.

The gene encoding the global regulatory protein irrE of the present disclosure may be obtained from the polynucleotide molecule encoding the global regulatory protein irrE comprising a partial nucleotide sequence of at least 100 consecutive nucleotides of SEQ ID NO: 1, preferably comprising a partial nucleotide sequence of at least 300 consecutive nucleotides of SEQ ID NO: 1, or more preferably comprising a partial nucleotide sequence of at least 600 consecutive nucleotides of SEQ ID NO: 1, and most preferably a polynucleotide comprising the nucleotide sequence of SEQ ID NO: 1. SEQ ID NO: 1 represents the whole nucleotide sequence of irrE isolated from Deinococcus radiodurans.

Said gene encoding the global regulatory protein irrE may also be obtained from a long polynucleotide sequence encoding the global regulatory protein irrE. Such polynucleotides may be isolated from bacteria, for example. Preferably, they are isolated from bacteria belonging to Deinococcus. Said bacteria include but are not limited to Deinococcus radiodurans, Deinococcus deserti, Deinococcus gobiensis and Deinococcus proteolyticus.

When such polynucleotides are obtained from a long polynucleotide sequence, it is possible to determine the homology between such polynucleotide sequence and SEQ ID NO: 1. In this case, preferably, a region having at least 100 consecutive nucleotides is selected and compared with the corresponding fragments derived from other polynucleotides. When the polynucleotide sequence has, for example, 60 nucleotides identical to the corresponding fragment obtainable from SEQ ID NO: 1 (by comparing 100 consecutive nucleotides), then the homology is 60%. Preferably, the partial polynucleotide sequence of the present disclosure has at least 80% homology and more preferably at least 90% homology with SEQ ID NO: 1. In order to determine the homology, for example, a fragment of at least 100 consecutive nucleotides, preferably a fragment of at least 300 consecutive nucleotides and more preferably a fragment of at least 500 consecutive nucleotides is used.

Those skilled in the art understand the fact described below. Some fragments in the polypeptide are necessary for the biological functions. However, there are other regions where an amino acid may be inserted, deleted, or substituted by other amino acids, preferably substituted by those amino acids similar to the amino acid being substituted.

Further, the object of the present disclosure is to provide a microorganism suitable for producing oxidized coenzyme Q10 with a high content. Patent document 5 discloses a fermentation method in which the content of oxidized coenzyme Q10 in coenzyme Q10 produced by the microorganism is increased by controlling the ORP of the fermentation broth. This publication is incorporated herein by reference.

We have found that, under suitable culture conditions, the microorganism having stress resistance may be used for further optimization of the direct production of oxidized coenzyme Q10 with a high content.

The terms “direct production”, “direct fermentation”, “direct transformation” and the like mean that the microorganism is capable of transforming a certain substrate into a specific product via one or more biological transformation steps without the need of any additional chemical transformation step, such as subjecting the reduced coenzyme Q10 obtained by extraction to further oxidization steps to obtain oxidized coenzyme Q10.

2. Preparation Method of the Recombinant Microorganism Used for Producing Coenzyme Q10

The inventor has genetically engineered the microorganism used for producing coenzyme Q10, so as to optimize the production of coenzyme Q10.

The preparation of the recombinant microorganism used for the production of coenzyme Q10 of the present disclosure includes the following steps:
step a. cloning a gene encoding the global regulatory protein irrE from a parent strain comprising the gene encoding the global regulatory protein irrE;
step b. ligating the gene encoding the global regulatory protein irrE to a vector, and constructing a recombinant vector comprising the gene encoding the global regulatory protein irrE; and
step c. constructing a recombinant microorganism comprising the gene encoding the global regulatory protein irrE by a method suitable for introducing the vector into a host cell, such as transformation, transduction, conjugative transfer and/or electroporation, and said host cell thus becoming the recombinant organism of the present disclosure.

In said step a, when the gene encoding the global regulatory protein irrE is isolated from a strain comprising the gene encoding the global regulatory protein irrE, the following exemplary methods may be adopted.

(i) The target gene is obtained by PCR using the primer designed based on the DNA sequence disclosed herein via methods known in the art.

(ii) After digesting the genome into several segments with restriction enzyme, the target gene is picked out by using a labeled nucleic acid probe.

(iii) The target gene is synthesized by methods known in the art, for example, using a DNA synthesizer.

Once a clone carrying the desired gene is obtained, the nucleotide sequence of the target gene may be determined by methods well known in the art.

In said step b, in the cloning of the double-stranded DNA, a series of combinations of host/cloning vector may be used. A preferred vector used for expressing the gene of the present disclosure (i.e., irrE gene) in E. coli may be selected from any of the vectors commonly used in E. coli, for example, pBR322 or the derivatives thereof (such as pUC18 and pBluescriptII (Stratagene Cloning Systems, Calif., USA)), pACYC177 and pACYC184 as well as the derivatives thereof, and vectors derived from broad-host-range plasmids such as RK2 and pBBR1MCS-2. A preferred vector used for expressing the nucleotide sequence of the present disclosure in Rhodobacter sphaeroides is selected from any of the vectors capable of being replicated in Rhodobacter sphaeroides and preferred cloning organisms (such as E. coli). A preferred vector is a broad-host-range vector, for example, a cosmid vector (such as pVK100) and the derivatives thereof, and pBBR1MCS-2. In a case where the properties of the host cell and the vector are taken into account, such vectors may be transferred into a preferred host by using any methods well known in the art such as transformation, transduction, conjugative transfer or electroporation.

The gene/nucleotide sequence of irrE provided by the present disclosure may be ligated to a suitable vector using methods well known in the art. Said vector comprises a regulatory sequence which is operable in the host cell, such as a promoter, a ribosome-binding site and a transcription terminator, so as to generate the recombinant vector.

In said step b, it is also possible to knockout the promoter in the recombinant vector in which said promoter controls the expression of the gene encoding the global regulatory protein irrE and then insert other different promoter(s) by promoter replacement, so as to further regulate the expression of the gene encoding the global regulatory protein irrE. The inserted promoter may be a constitutive promoter or an inducible promoter, for example, an original promoter of a gene, a promoter of an antibiotic resistance gene, an osmoregulated promoter, a temperature-inducible promoter, a beta-galactosidase (lac), trp, tac, trc promoter of E. coli, and any promoter capable of functioning in the host cell. Preferably, said promoter is an inducible promoter, in particular, an osmoregulated promoter, and more preferably, an osmoregulated promoter proPB.

Said osmoregulated promoter proPB may be obtained from the polynucleotide molecule of the osmoregulated promoter proPB comprising a partial nucleotide sequence of at least 70 consecutive nucleotides of SEQ ID NO: 2, preferably comprising a partial nucleotide sequence of at least 100 consecutive nucleotides of SEQ ID NO: 2, more preferably comprising a partial nucleotide sequence of at least 150 consecutive nucleotides of SEQ ID NO: 2, and most preferably comprising the polynucleotides of the nucleotide sequence of SEQ ID NO: 2. SEQ ID NO: 2 represents the whole nucleotide sequence of the proPB promoter isolated from Escherichia coli.

Said osmoregulated promoter proPB may also be obtained from a long polynucleotide sequence comprising the osmoregulated promoter proPB. Such polynucleotides may be for example isolated from bacteria, preferably Escherichia, and more preferably Escherichia coli. When such polynucleotides are obtained from a long polynucleotide sequence, it is possible to determine the homology between such polynucleotide sequence and SEQ ID NO: 2. Hereinafter, the definition of homology is same as that of the aforementioned SEQ ID NO: 1. Preferably, the partial polynucleotide sequence of the present disclosure has at least 80% homology and more preferably at least 90% homology with SEQ ID NO: 2. In order to determine the homology, for example, a fragment of at least 100 consecutive nucleotides and preferably a fragment of at least 200 consecutive nucleotides is used.

For the purpose of expression, other regulatory elements, for example, the Shine-Dalgarno (SD) sequence operable in the host cell (a coding sequence will be introduced therein to provide the recombinant cell of the present disclosure) (eg. AGGAGG and the like, including natural and synthetic sequences that are operable in the host cell), and a transcription terminator (inverted repeat structure including any natural and synthetic sequences), may be used together with the promoter described above.

In said step c, in order to construct a recombinant microorganism carrying the recombinant vector, a variety of gene transfer methods such as transformation, transduction, conjugative transfer or electroporation may be used. The method for constructing a recombinant cell may be selected from methods well known in the field of molecular biology. For example, a conventional transformation system may be used in Escherichia coli. A transduction system may also be used in Escherichia coli, and a conjugative transfer system may be widely used in Gram-positive and Gram-negative bacteria, such as Escherichia coli and Rhodobacter sphaeroides. CN103509816B discloses a conjugative transfer method, wherein the conjugation may occur in a liquid medium or on the surface of a solid medium. A selective marker may be added to the receptor used for conjugative transfer, for example, a kanamycin-resistant marker is commonly selected. A natural resistant marker may also be used, for example, a nalidixic acid-resistant marker may be used in Rhodobacter sphaeroides.

The present disclosure also relates to a recombinant vector comprising said polynucleotides, preferably a recombinant vector capable of functioning in a suitable host cell.

Microorganisms conventionally used for the production of coenzyme Q10 in the art, including any one of bacteria, yeast, and mold, may be used in the present disclosure, and the above-mentioned recombinant microorganism of the present disclosure may be obtained after applying genetic engineering techniques well known in the art to said microorganisms. Specifically, said microorganisms include microorganisms such as Agrobacterium, Agromonas, Brevundimonas, Pseudomonas, Rhodotorula, Rhizomonas, Rhodobium, Rhodoplanes, Rhodopseudomonas, Rhodobacter, Rhizobium, and the like, preferably, Agrobacterium tumefacience, Agrobacterium radiobacter, Agromonas oligotrophica, Brevundimonas diminuta, Pseudomonas denitrificans, Rhodotorula minuta, Rhodopseudomonas palustris, Phodobacter capsulatus, Rhodobacter sphaeroides, and the like, and further preferably Rhodobacter sphaeroides.

Therefore, the present disclosure also relates to the host cell as described above, wherein said host cell has a recombinant vector comprising said polynucleotides. Such host cells after modified by genetic engineering are referred to as recombinant host cells or recombinant microorganisms.

3. Producing Coenzyme Q10 by Microbial Fermentation

The microbial fermentation method used for the production of coenzyme Q10 in the present disclosure is characterized by using the above-mentioned recombinant microorganism to carry out fermentation production. Since the recombinant microorganism of the present disclosure may, on one hand, enhance the tolerance of the microorganism against a variety of stresses such as osmotic pressure, oxidation, radiation and heat, as compared with the fermentation methods of coenzyme Q10 in the prior art, on the one hand, the logarithmic growth phase of the strain is prolonged and further accumulation of biomass is promoted, on the other hand, the growth and metabolism of the strain are vigorous, which significantly increases the potency of coenzyme Q10. In the method of the present disclosure, the conditions of the fermentation technology used in the production of coenzyme Q10 by using a recombinant microorganism may be referred to Patent document 1. The specific method is as follows.

During the fermentation process of the coenzyme Q10-producing strain, the oxygen consumption rate is maintained between 30 mmol/L·h and 150 mmol/L·h while the electrical conductivity is maintained between 5.0 ms/cm and 30.0 ms/cm, so as to facilitate the growth of the microbe as well as the initiation of the coenzyme Q10 synthesis and the accumulation of coenzyme Q10. Preferably, during the fermentation process of the coenzyme Q10-producing strain, the oxygen consumption rate is controlled between 30 mmol/L·h and 90 mmol/L·h. Preferably, during the fermentation process of the coenzyme Q10-producing strain, the electrical conductivity of the fermentation broth is controlled between 10 ms/cm and 20 ms/cm.

In the fermentation production method of coenzyme Q10 of the present disclosure, said oxygen consumption rate is adjusted by the agitation speed and the airflow rate, and said electrical conductivity is adjusted by flow feeding or batch feeding. Among them, the formula of the feeding liquid used in flow feeding or batch feeding is as follows: in terms of one liter of feeding liquid, 8 to 12 g of yeast powder, 5 to 10 g of (NH₄)₂SO₄, 1 to 2 g of MgSO₄, 3 to 6 g of NaCl, 2 to 4 g of KH₂PO₄, 2 to 4 g of K₂HPO₄, 1 to 2 g of CaCl₂, 0.013 to 0.025 g of biotin. The pH value is 7.0, and the electrical conductivity of the feeding medium is 13.5 ms/cm to 23 ms/cm.

In the fermentation production method of coenzyme Q10 of the present disclosure, it is possible to use not only Rhodobacter sphaeroides RSP-BE but also strains breeded by physical or chemical mutagenesis methods or strains modified by genetic engineering methods.

The method of the present disclosure enables the potency of coenzyme Q10 to be at least 1000 mg/L, preferably at least 2000 mg/L, and more preferably at least 3000 mg/L. The potency of coenzyme Q10 refers to the content of coenzyme Q10 per unit volume of the fermentation broth.

As could be seen from the contents described above, the recombinant microorganism of the present disclosure has obvious advantages in increasing the potency of coenzyme Q10 even if a conventional fermentation method of coenzyme Q10 in the art is used. Accordingly, the recombinant microorganism of the present disclosure is suitable for the conventional fermentation technologies of coenzyme Q10 in the art.

4. Producing Oxidized Coenzyme Q10 with a High Content by Microbial Fermentation

As described above, as compared with the prior art, the improvement of the recombinant microorganism of the present disclosure also lies in being capable of further increasing the yield of oxidized coenzyme Q10.

By using specific recombinant microorganism, the method of the present disclosure may significantly enhance the tolerance against harsh environments including high osmotic pressure and high redox potential, eliminate the adverse effects on microbe exerted by high redox potential in the fermentation production method of oxidized coenzyme Q10, further exert the facilitation of oxidative stress on coenzyme Q10 production carried out by using the microbe, and increase the potency of oxidized coenzyme Q10. In the method of the present disclosure, the conditions of the fermentation technology used in the production of oxidized coenzyme Q10 by using the fermentation of a recombinant microorganism may be referred to Patent document 5. The specific method is as follows.

Provided is a fermentation production method of oxidized coenzyme Q10, wherein the ORP of the fermentation broth is controlled at the synthesis and accumulation stage of coenzyme Q10 during the fermentation process. Preferably, during the fermentation process, the ORP of the fermentation broth is controlled in the middle and later periods of the synthesis and accumulation stage of coenzyme Q10. It is also preferred to control the ORP of the fermentation broth in the later period of the synthesis and accumulation stage of coenzyme Q10 during the fermentation process. During the fermentation process of the producing strain, the oxidation-reduction potential ORP of the fermentation broth is controlled to be −50 to 300 mV, preferably, the oxidation-reduction potential ORP of the fermentation broth is controlled to be 50 to 200 mV.

In the above-mentioned fermentation production method, during the fermentation process described above, the electrical conductivity of the fermentation broth is controlled between 5.0 ms/cm and 30.0 ms/cm. Preferably, in the growth stage of the microbe, the oxygen consumption rate is controlled between 30 mmol/(L·h) and 150 mmol/(L·h), and the electrical conductivity of said fermentation broth is controlled between 5.0 ms/cm and 30.0 ms/cm. It is also preferred that, at the synthesis and accumulation stage of coenzyme Q10, the oxygen consumption rate is controlled between 60 mmol/(L·h) and 120 mmol/(L·h), and the electrical conductivity of said fermentation broth is controlled between 8.0 ms/cm and 15.0 ms/cm.

In the above-mentioned fermentation production method, the oxidation-reduction potential ORP of the fermentation broth is controlled by at least one of the following ways: controlling the dissolved oxygen in said fermentation broth, and controlling the pH of said fermentation broth. It is preferred to combine the way of controlling the dissolved oxygen in said fermentation broth and the way of controlling the pH of said fermentation broth.

In the above-mentioned fermentation production method, the dissolved oxygen in said fermentation broth is controlled by at least one of the following ways: controlling the input power of stirring per unit volume of the fermentation tank, controlling the air intake flow rate per unit volume of the fermentation broth, and controlling the internal pressure of the fermentation tank. It is preferred to combine two or more of the ways described above to control the dissolved oxygen in said fermentation broth.

In the above-mentioned fermentation production method, at the synthesis and accumulation stage of coenzyme Q10, said input power of stirring per unit volume of the fermentation tank is preferably between 0.25 kw/m³ and 0.50 kw/m³, said air intake flow rate per unit volume of the fermentation broth is preferably between 1.0 vvm and 15.0 vvm, and/or said internal pressure of the fermentation tank is preferably between 0.05 MPa and 0.3 MPa; more preferably, said input power of stirring per unit volume of the fermentation tank is between 0.30 kw/m³ and 0.40 kw/m³, said air intake flow rate per unit volume of the fermentation broth is between 5.0 vvm and 8.0 vvm, and/or said internal pressure of the fermentation tank is between 0.08 MPa and 0.15 MPa.

In the above-mentioned fermentation production method, at the synthesis and accumulation stage of coenzyme Q10, the pH of said fermentation broth is controlled by controlling the pH of said fermentation broth between 3.5 and 6.0. Preferably, the pH of said fermentation broth is controlled by controlling the pH of said fermentation broth between 4.0 and 5.0. It is also preferred to control the pH of said fermentation broth by way of adding an acid or adding an alkali. It is further preferred to control the pH of said fermentation broth by way of adding said acid or said alkali by stages or continuously.

In the above-mentioned fermentation production method, said acid is an organic acid or an inorganic acid, and/or said alkali is an organic alkali or an inorganic alkali; preferably, said acid is one or two or more of phosphoric acid, hydrochloric acid, sulfuric acid, lactic acid, propionic acid, citric acid and oxalic acid, and/or said alkali is preferably one or two or more of aqueous ammonia, sodium hydroxide and liquid ammonia; and more preferably, said acid is phosphoric acid, lactic acid or citric acid, and/or said alkali is aqueous ammonia or liquid ammonia.

In the above-mentioned fermentation production method, it is possible to use not only Rhodobacter sphaeroides RSP-BE but also strains bred by physical or chemical mutagenesis methods or strains modified by genetic engineering methods.

In the above-mentioned fermentation production method, said coenzyme Q10 of the production has a higher content of oxidized coenzyme Q10. The content of oxidized coenzyme Q10 is preferably 96% or more, more preferably 97% or more, and most preferably 99% or more.

In the above-mentioned fermentation production method, said oxidized coenzyme Q10 has a potency of at least 1000 mg/L, preferably at least 2000 mg/L, and more preferably at least 3000 mg/L. The potency of oxidized coenzyme Q10 refers to the content of oxidized coenzyme Q10 per unit volume of the fermentation broth.

The oxidized coenzyme Q10 obtained by the above-mentioned fermentation production method may be used to prepare all kinds of foods including functional nutritive foods and special health foods, and may also be used to prepare nutritional supplements, nourishments, animal medicines, beverages, feed, cosmetics, drugs, medicaments, and preventive medicines.

The present disclosure is described in detail below with reference to the drawings and examples, and the present disclosure is not limited thereto.

### EXAMPLES

The construction of the recombinant microorganisms in the examples of the present application included the following basic operations.

The media used in the present disclosure were as follows.

The formula of the slant culture medium (100 ml) was 0.8 g of yeast extract, 0.01 g of FeSO₄, 0.13 g of K₂HPO₄, 0.003 g of CoCl₂, 0.2 g of NaCl, 0.0001 g of MnSO₄, 0.025 g of MgSO₄, 0.3 g of glucose, 0.1 μg of Vitamin B1, 0.1 μg of Vitamin K, 0.15 μg of Vitamin A, and 1.5 g of agar powder, and the pH was adjusted to 7.2.

The formula of the seed culture medium (100 ml) was 0.25 g of (NH₄)₂SO₄, 0.05 g of corn steep liquor, 0.14 g of yeast extract, 0.2 g of NaCl, 0.3 g of glucose, 0.05 g of K₂HPO₄, 0.05 g of KH₂PO₄, 0.1 g of MgSO₄, 0.01 g of FeSO₄, 0.003 g of CoCl₂, 0.0001 g of MnSO₄, 0.8 g of CaCO₃, 0.1 μg of Vitamin B1, 0.1 μg of Vitamin K, and 0.15 μg of Vitamin A, and the pH was adjusted to 7.2.

The formula of the fermentation culture medium (100 ml) was 0.3 g of (NH₄)₂SO₄, 0.28 g of NaCl, 4 g of glucose, 0.15 g of KH₂PO₄, 0.3 g of monosodium glutamate, 0.63 g of MgSO₄, 0.4 g of corn steep liquor, 0.12 g of FeSO₄, 0.005 g of CoCl₂, 0.6 g of CaCO₃, 0.1 μg of Vitamin B1, 0.1 μg of Vitamin K, and 0.15 μg of Vitamin A, and the pH was adjusted to 7.2.

The determination of the potency was as follows.

Sample preparation: Under a nitrogen atmosphere, 1 ml of the fermentation broth was taken and transferred into a 10-ml centrifuge tube and 180 μl of 1 mol/L HCl was added. The mixture was well mixed, allowed to stand still for 3 min to 5 min, and then placed in a water bath of 92° C. and heated for 30 min. The mixture was centrifuged to remove the supernatant, 8 ml of leaching liquor (ethyl acetate:ethanol=5:3) was added, the extraction lasted for 2 h, and the resulting mixture was tested by reverse-phase HPLC. HPLC conditions: C18 column: 150 mm×4.6 mm, mobile phase: methanol:isopropanol=75:25 (in terms of volume), flow rate: 1.00 ml/min, detection wavelength: 275 nm, injection volume: 40 μl, retention time: 12 min.

The content of oxidized coenzyme Q10 was determined as follows.

Sample preparation: Under a nitrogen atmosphere, 1 ml of the fermentation broth was taken and transferred into a 10-ml centrifuge tube and 180 μl of 1 mol/L HCl was added. The mixture was well mixed, allowed to stand still for 3 min to 5 min, and then placed in a water bath of 92° C. and heated for 30 min. The mixture was centrifuged to remove the supernatant, 8 ml of leaching liquor (ethyl acetate:ethanol=5:3) was added, the extraction lasted for 2 h, and the resulting mixture was tested by reverse-phase HPLC. HPLC conditions: column: YMC-Pack 250 mm×4.6 mm, mobile phase: methanol/n-hexane=85:15 (in terms of volume), flow rate: 1 mL/min, detection wavelength: 275 nm, injection volume: 40 μl, retention time: 13.5 min for reduced coenzyme Q10, 22.0 min for oxidized coenzyme Q10.

The determination of biomass was as follows. 10 ml of the fermentation broth was taken and weighed, 2 mol/L of hydrochloric acid solution was added, and the pH adjusted to about 4.0. The mixture was kept at 80° C. for 20 min, centrifuged to discard the supernatant, washed with water, centrifuged to discard the supernatant, and dried at 60° C. for 20 hours. The resultant was weighed, and the microbe content in each kilo of the fermentation broth was calculated.

Technical means well known in the art such as a determination method using glucose analyzer may be used for the determination of the residual glucose. Technical means well known in the art such as the molybdenum blue colorimetry may be used for the determination of the dissolved phosphate.

### Example 1 Amplification of the Gene Encoding the Global Regulatory Protein irrE and Construction of a Recombinant Vector

The genome of Deinococcus radiodurans was extracted (the reagents were obtained from Ezup Column Bacteria Genomic DNA Purification Kit produced by Sangon Biotech (Shanghai) Co., Ltd.), and the extraction process was conducted according to the instructions attached to the kit.

According to the DNA sequence set forth in SEQ ID NO: 1, the primer design software Primer 5 was used to design and obtain the following primers: the forward primer irrE-F, i.e., 5′-ccgGAATTCGTGCCCAGTGCCAACGTCAGCCCCCCTTG-3′ (the underlined part was an EcoRI restriction site) as SEQ ID No. 3, and the reverse primer irrE-R, i.e., 5′-cgcGGATCCTCACTGTGCAGCGTCCTGCGGCTCGTC-3′ (the underlined part was a BamHI restriction site) as SEQ ID No. 4.

The genomic DNA extracted from Deinococcus radiodurans was used as a template, high-fidelity PrimeSTAR DNA Polymerase (purchased from Dalian Takara Bio Corporation) and the primers as shown by SEQ ID No. 3 and SEQ ID No. 4 were used, and a PCR method was adopted to synthesize the gene of the global regulatory protein irrE. The following standard reaction system was adopted.

| | | |
|---|---|---|
| GC buffer | 25 | μl |
| Water | 16 | μl |
| dNTP | 4 | μl |
| Forward primer | 1.5 | μl (10 μM) |
| Reverse primer | 1.5 | μl (10 μM) |
| Genomic DNA of Deinococcus radiodurans | 1.5 | μl |
| PrimeSTAR DNA polymerase | 0.5 | μl |
| Total | 50 | μl |

The amplification procedure included 30 cycles, and each cycle included denaturation at 98° C. for 10 seconds, annealing at 55° C. for 15 seconds and extension at 72° C. for 1 minute.

The PCR product was taken out and subjected to PCR purification (the reagents were obtained from Axygen PrepPCR Clean-up Kit), the purification process was conducted according to the instructions attached to the kit, and the product resulting from PCR purification was obtained.

Enzyme digestion was performed in accordance with the endonuclease standard system of Takara Corporation. The standard system was as follows.

| | |
|---|---|
| EcoRI | 1 μl |
| BamHI | 1 μl |
| 10 × buffer | 3 μl |
| Product resulting from PCR purification | 25 μl |
| Total | 30 μl |
| EcoRI | 1 μl |
| BamHI | 1 μl |
| 10 × buffer | 3 μl |
| pBBR1MCS-2 plasmid | 25 μl |
| Total | 30 μl |

The enzymatic digested product was taken out and subjected to gel extraction (the reagents were obtained from Axygen PrepDNA Gel Extraction Kit), the extraction process was conducted according to the instructions attached to the kit, and the extracted gene fragments were obtained.

According to the instructions of T4 ligase produced by Takara Corporation and in accordance with the standard system, 5.5 μl of irrE gene obtained from gel extraction, 3 μl of plasmid pBBR1MCS-2 obtained from gel extraction, 0.5 μl of T4 ligase and 1 μl of T4 ligase BUFFER were mixed and ligated in a water bath of 22° C. for 60 minutes to obtain a recombinant plasmid pBBR1MCS-2-irrE.

### Example 2 the Substitution of Osmoregulated Promoter proPB

The recombinant vector pBBR1MCS-2-irrE was transformed into Escherichia coli BL21 competent cells by heat-shock method, colonies capable of growing on an LB plate medium containing 50 μg/ml of kanamycin were cultured consecutively on this LB plate medium, and a genetically stable recombinant Escherichia coli was obtained.

The plasmid of the genetically stable recombinant Escherichia coli was extracted (the reagents were obtained from AxyPrep plasmid DNA Miniprep Kit), and the extraction process was conducted according to the instructions attached to the kit.

PCR verification was conducted by using primers irrE-F and irrE-R, and a fragment of approximately 1.0 kb was obtained, indicating that the gene encoding the global regulatory protein irrE had been successfully introduced into the recombinant Escherichia coli.

The forward primer lac-F, i.e., 5′-GCCTGGGGTGCCTAATGAG TGAGCTAACTCACATTAATTGCG-3′ (the underlined part was the homologous sequence) as SEQ ID No. 5 and the reverse primer lac-R, i.e., 5′-CTCATTAGGCACCCCAGGC TGTGGAATTGTGAGCGGATAACAATTTC-3′ (the underlined part was the homologous sequence) as SEQ ID No. 6 were designed.

The plasmid DNA of the recombinant Escherichia coli verified by PCR was used as a template, high-fidelity PrimeSTAR DNA polymerase of Takara Corporation (Dalian Takara Bio Corporation) and a recommended system were used, and the amplification was conducted by circularized PCR method using the primers as shown by SEQ ID No. 5 and SEQ ID 6. A standard reaction system was adopted.

| | | |
|---|---|---|
| GC buffer | 12.5 | μl |
| Water | 7 | μl |
| dNTP | 2 | μl |
| Forward primer | 1.0 | μl (10 μM) |
| Reverse primer | 1.0 | μl (10 μM) |
| Plasmid DNA of the recombinant Escherichia coli | 1.0 | μl |
| PrimeSTAR DNA polymerase | 0.5 | μl |
| Total | 25 | μl |

The amplification procedure included 20 cycles, and each cycle included denaturation at 98 □for 10 seconds, annealing at 55° C. for 15 seconds and extension at 72° C. for 6 minutes.

The PCR product was taken out and subjected to PCR purification (the reagents were obtained from Axygen PrepPCR Clean-up Kit), the purification process was conducted according to the instructions attached to the kit, and the product resulting from PCR purification was obtained.

The product resulting from PCR purification was transformed into Escherichia coli BL21 competent cells by heat-shock method, colonies capable of growing on an LB plate medium containing 50 μg/ml of kanamycin were cultured consecutively on this LB plate medium, and a genetically stable recombinant Escherichia coli was obtained.

The plasmid of the genetically stable recombinant Escherichia coli was extracted (the reagents were obtained from AxyPrep plasmid DNA Miniprep Kit), the extraction process was conducted according to the instructions attached to the kit, and a recombinant plasmid pBBR1MCS-2-G-irrE in which the lac promoter was knocked out was obtained.

It was confirmed by the plasmid sequencing and verification conducted by Sangon Biotech (Shanghai) Co., Ltd. that the lac promoter had been knocked out and the gene sequence of irrE was accurate. The genome of Escherichia coli was extracted (the reagents were obtained from Ezup Column Bacteria Genomic DNA Purification Kit produced by Sangon Biotech (Shanghai) Co., Ltd.), and the extraction process was conducted according to the instructions attached to the kit.

The forward primer proPB-F, i.e., 5′-ccgCTCGAGCATGTGTGAAGTTGATCAC AAATTT-3′ (the underlined part was an XhoI restriction site) as SEQ ID No. 7, and the reverse primer proPB-R, i.e., 5′-cccAAGCTTGAGTTGGCCCATTTCCGCAAACG-3′ (the underlined part was a HindIII restriction site) as SEQ ID No. 8 were designed.

The genomic DNA extracted from Escherichia coli was used as a template, high-fidelity PrimeSTAR DNA polymerase (purchased from Dalian Takara Bio Corporation) and the primers as shown by SEQ ID No. 7 and SEQ ID No. 8 were used, and a PCR method was adopted to synthesize the gene of the global regulatory protein irrE. The following standard reaction system was adopted.

| | | |
|---|---|---|
| GC buffer | 25 | μl |
| Water | 16 | μl |
| dNTP | 4 | μl |
| Forward primer | 1.5 | μl (10 μM) |
| Reverse primer | 1.5 | μl (10 μM) |
| Genomic DNA of Escherichia coli | 1.5 | μl |
| PrimeSTAR DNA polymerase | 0.5 | μl |
| Total | 50 | μl |

The amplification procedure included 30 cycles, and each cycle included denaturation at 98° C. for 10 seconds, annealing at 55° C. for 15 seconds and extension at 72° C. for 1 minute.

The PCR product was taken out and subjected to PCR purification (the reagents were obtained from Axygen PrepPCR Clean-up Kit), the purification process was conducted according to the instructions attached to the kit, and the product resulting from PCR purification was obtained.

Enzyme digestion was performed in accordance with the endonuclease standard system of Takara Corporation. The standard system was as follows.

| | |
|---|---|
| XhoI | 1 μl |
| HindIII | 1 μl |
| 10 × buffer | 3 μl |
| Product resulting from PCR purification | 25 μl |
| Total | 30 μl |
| XhoI | 1 μl |
| HindIII | 1 μl |
| 10 × buffer | 3 μl |
| pBBR1MCS-2-G-irrE plasmid | 25 μl |
| Total | 30 μl |

The enzymatic digested product was taken out and subjected to gel extraction (the reagents were obtained from Axygen PrepDNA Gel Extraction Kit), the extraction process was conducted according to the instructions attached to the kit, and the extracted gene fragments were obtained.

According to the instructions of T4 ligase produced by Takara Corporation and in accordance with the standard system, 5.5 μl of proPB sequence obtained from gel extraction, 3 μl of plasmid pBBR1MCS-2-G-irrE obtained from gel extraction, 0.5 μl of T4 ligase and 1 μl of T4 ligase BUFFER were mixed and ligated in a water bath of 22° C. for 60 minutes, and a recombinant plasmid pBBR1MCS-2-G-proPB-irrE was obtained, as shown by FIG. 2.

### Example 3 Construction of a Recombinant Microorganism Comprising the Gene Encoding the Global Regulatory Protein irrE and the Osmoregulated Promoter proPB

The recombinant vector pBBR1MCS-2-G-proPB-irrE was transformed into Escherichia coli S17-1 competent cells by heat-shock method, colonies capable of growing on an LB plate medium containing 50 μg/ml of kanamycin were cultured on this LB plate medium, and a recombinant Escherichia coli was obtained. The recombinant Escherichia coli was picked and the genome was extracted. PCR verification was conducted by using primers proPB-F and irrE-R, and a fragment of approximately 1.2 kb was obtained, indicating that the proPB promoter and the gene encoding the global regulatory protein irrE had been successfully introduced into the recombinant Escherichia coli. Also, it was confirmed by the sequencing and verification conducted by Sangon Biotech (Shanghai) Co., Ltd. that the sequence was consistent with the sequence in NCBI.

The recombinant vector pBBR1MCS-2-G-proPB-irrE in the recombinant Escherichia coli was introduced into Rhodobacter sphaeroides by conjugative transfer, Rhodobacter sphaeroides capable of growing on a plate medium containing 50 μg/ml of nalidixic acid and kanamycin was inoculated and transformed for three consecutive generations on this plate medium, and a genetically stable recombinant Rhodobacter sphaeroides RSP-BE was obtained.

The recombinant Rhodobacter sphaeroides was picked and the genome was extracted. PCR verification was conducted by using primers proPB-F and irrE-R, and a fragment of approximately 1.2 kb was obtained, indicating that the proPB promoter and the gene encoding the global regulatory protein irrE had been successfully introduced into the recombinant Rhodobacter sphaeroides RSP-BE.

During the construction process of the above-mentioned recombinant microorganism, the specific operations of transforming the recombinant vector into Escherichia coli S17-1 were as follows.

The tube containing Escherichia coli S17-1 competent cells was taken out and placed in an ice bath for 10 minutes. After that, the recombinant plasmid pBBR1MCS-2-G-proPB-irrE was added thereto. The tube was placed in an ice bath for 20 minutes, subjected to heat shock for 90 seconds, and placed in an ice bath for 5 minutes. 600 μl of LB liquid medium was added thereto. After incubated at 37° C. for 45 minutes, the mixture was centrifuged at 5000 rpm for 5 minutes. 500 μl of the supernatant was discarded, and the remaining liquid was smeared on a plate medium containing kanamycin.

The specific operations of conjugative transfer were as follows.

Rhodobacter sphaeroides was inoculated in a test tube containing 10 ml of liquid medium and cultured under a condition of 30 □ and 200 rpm for 50 h.

After 32 hours, the positive clones of the transformed Escherichia coli S17-1 were inoculated to LB liquid medium and cultured under a condition of 37 □ and 200 rpm overnight. After 15 hours, Escherichia coli S17-1 was inoculated and transformed. In each test tube, 100 μl of the bacteria solution was added to 5 ml of LB medium, 5 μl of kanamycin was added, and the test tube was placed in a shaker at 37° C. for cultivation. After being cultured for 3 to 4 hours, 4 ml of Rhodobacter sphaeroides bacteria solution and 2 ml of Escherichia coli bacteria solution were taken and separately dispensed into 2-ml centrifuge tubes with 1 ml in each centrifuge tube, and the centrifuge tubes were centrifuged at 5000 rpm for 5 minutes. The supernatants were discarded separately, 1 mL of fresh LB medium was added, the bacteria were resuspended gently, and the resulting mixture was centrifuged at 5000 rpm for 5 minutes. The supernatants were discarded separately, 1 mL of fresh LB medium was added, and the bacteria were resuspended gently. The bacteria solution was mixed evenly with a ratio of Rhodobacter sphaeroides to Escherichia coli being 100:50 or 100:100, a filter membrane (0.22 μm) was pasted in the center of the LB plate, and the mixed bacteria solution was poured into the central region of the filter membrane. The LB plate was carefully transferred to an incubator at 32 □° C. and incubated overnight.

The filter membrane was transferred to a 2-mL EP tube with a tweezer. After that, the bacteria on the filter membrane were washed with 500 μl of LB liquid medium, dispersed, respectively dispensed and smeared on the plate medium with each plate comprising 350 μl of bacteria solution, and then placed in an incubator at 32° C. and cultured for 72 hours.

Whether a clone was a positive one was tested after the conjugative transfer.

2 to 5 well-grown colonies were picked and cultured for 48 to 60 hours, inoculated and transformed, and cultured for another 2 to 4 hours. After that, the plasmid was extracted in accordance with the operation steps in the instructions of Axygen Plasmid Extraction Kit. A centrifuge tube was taken, 34 μl of the plasmid obtained from the extraction in step 2 was added, 1 μl of XhoI and 1 μl of BamHI were added, and 4 μl of BUFFER was added. The centrifuge tube was placed in a water bath of 37° C. and the enzyme digestion lasted for 1.5 hours. After the enzyme digestion, the resulting mixture was subjected to electrophoresis detection, and a clear band around 1.2 kb was shown by electrophoresis, which was in line with the expectation. The obtained DNA fragments were subjected to sequencing and verification after gel extraction, and were confirmed as positive clones.

The strain obtained by this method was subjected to culture preservation. This bacterium was Rhodobacter sphaeroides with a Latin scientific name of Rhodobacter sphaeroides, and was named as RSP-BE strain. It was preserved in China General Microbiological Culture Collection Center (CGMCC, Institute of Microbiology, Chinese Academy of Sciences, NO. 1 West Beichen Road, Chaoyang District, Beijing, postcode: 100101) on Jun. 11, 2018, and the preservation number was CGMCC No. 15927.

### Example 4 Producing Coenzyme Q10 by Using the Fermentation of the Recombinant Microorganism

A single colony of the recombinant Rhodobacter sphaeroides RSP-BE that had been cultured on a plate for about 7 days was selected, correspondingly picked and transferred to the slant culture medium in a small test tube for cultivation. The slant on which the bacteria were cultured was washed with sterile water, and a bacterial suspension having a bacteria concentration of 10⁸ to 10⁹ cells per milliliter was prepared. The prepared bacterial suspension was inoculated into a seed medium with an inoculation amount of 2% and was subjected to seed culture, wherein the volume of the medium was 100 ml, the temperature was 32° C., the rotating speed was 180 rpm, and the mixture was cultured for 22 to 26 hours.

The Rhodobacter sphaeroides strain CGMCC No. 15927 obtained after the seed culture was inoculated into a 10-L fermentation tank with an inoculation amount of 10%. The inoculation amount could be a conventional content in the art, for example, 1% to 30%, preferably 2.5% to 20%, and still preferably 5% to 15%. The inoculation amount could be adjusted as required.

The fermentation of the seed liquid was initiated in a 10-L fermentation tank, the fermentation temperature was 31 □, the pressure in the tank was 0.03 MPa, and a strategy of controlling the oxygen supply by stages was adopted. From 0 to 24 hours, the stirring speed was controlled to be 500 rpm and the air flow rate was controlled to be 6 L/min. With the growth of the bacteria, OUR slowly became stable and reached 50 mmol/L·h. At this stage, the bacteria were still in the exponential growth phase, and the oxygen supply had become a growth-limiting condition. The oxygen supply level was improved by increasing the stirring speed and the volume of aeration. OUR was maintained at 60 mmol/L·h from 24 to 36 hours and was maintained at 70 mmol/L·h from 36 to 60 hours, so as to promote the growth of bacteria. After 60 hours, the bacteria of this stage gradually entered a stable phase, the number of bacteria no longer increased, and coenzyme Q10 was synthesized and accumulated rapidly. The oxygen supply was gradually reduced to maintain a high specific production rate of coenzyme Q10. OUR was maintained at 90 mmol/L·h from 60 to 90 hours, maintained at 80 mmol/L·h from 90 to 100 hours, and maintained at 60 mmol/L·h after 100 hours.

Control of the feeding technology during the fermentation process. In addition to feeding glucose and potassium dihydrogen phosphate in the fermentation technology based on the residual glucose and the dissolved phosphate as well known in the art, in the present disclosure, the flow feeding of the medium started when the electrical conductivity dropped to 15.0 ms/cm. The formula of the feeding medium was as follows. Each liter of the feeding liquid contained 12 g of yeast powder, 10 g of (NH₄)₂SO₄, 2 g of MgSO₄, 6 g of NaCl, 4 g of KH₂PO₄, 4 g of K₂HPO₄, 2 g of CaCl₂, and 0.025 g of biotin, and the pH value was 7.0. The feeding rate of the medium was controlled such that the electrical conductivity was maintained within 15 ms/cm, and the residual glucose was maintained at 2.0% during the whole process. After 110 hours when the fermentation was completed, partial fermentation broth was taken, and extracted and tested under an inert gas atmosphere. The measured potency was 3637 mg/L, and the biomass was 125 g/kg.

### Example 5 Producing Oxidized Coenzyme 010 by Using the Fermentation of the Recombinant Microorganism

A single colony of the recombinant Rhodobacter sphaeroides RSP-BE that had been cultured on a plate for about 7 days was selected, correspondingly picked and transferred to the slant culture medium in a small test tube for cultivation. The slant on which the bacteria were cultured was washed with sterile water, and a bacterial suspension having a bacteria concentration of 10⁸ to 10⁹ cells per milliliter was prepared. The prepared bacterial suspension was inoculated into a seed medium with an inoculation amount of 2% and was subjected to seed culture, wherein the volume of the medium was 100 ml, the temperature was 32° C., the rotating speed was 180 rpm, and the mixture was cultured for 22 to 26 hours.

The Rhodobacter sphaeroides strain CGMCC No. 15927 obtained after the seed culture was inoculated into a 10-L fermentation tank with an inoculation amount of 10%. The inoculation amount could be a conventional content in the art, for example, 1% to 30%, preferably 2.5% to 20%, and still preferably 5% to 15%. The inoculation amount could be adjusted as required.

The fermentation of the seed liquid was initiated in a 10-L fermentation tank, the fermentation temperature was 30° C., the air intake flow rate per unit volume of the fermentation broth in the fermentation tank was controlled to be 0.4 vvm, the input power of stirring per unit volume was controlled to be 0.1 kw/m³, the tank pressure was 0.02 MPa, the oxygen consumption rate was controlled to be 50 mmol/(L·h), the electrical conductivity of the fermentation broth was controlled to be 12 ms/cm, and the pH value was controlled to about 7.0.

The feeding medium contained 12 g of yeast powder, 10 g of (NH₄)₂SO₄, 2 g of MgSO₄, 6 g of NaCl, 4 g of KH₂PO₄, 4 g of K₂HPO₄, 2 g of CaCl₂) and 0.025 g of biotin in each liter of the feeding liquid, and the pH value was adjusted to 7.0.

After 15 hours, the oxygen supply was increased, the air intake flow rate per unit volume of the fermentation broth in the fermentation tank was controlled to be 0.6 vvm, the input power of stirring per unit volume was controlled to be 0.2 kw/m³, the tank pressure was 0.04 MPa, the oxygen consumption rate was kept steady after rising to 70 mmol/(L·h), the electrical conductivity of the fermentation broth was controlled to be 12 ms/cm, the pH value was controlled to be 7.0, and the fermentation was continued. The fermentation at this time was within the growth stage of the bacteria.

After 20 hours, the oxygen supply was increased again, the air intake flow rate per unit volume of the fermentation broth in the fermentation tank was controlled to be 0.8 vvm, the input power of stirring per unit volume was controlled to be 0.2 kw/m³, the tank pressure was 0.05 MPa, the oxygen consumption rate was kept steady after rising to 90 mmol/(L·h), the electrical conductivity of the fermentation broth was controlled to be 12 ms/cm, the pH value was controlled to be 7.0, and the fermentation was continued. The fermentation at this time was within the growth stage of the bacteria.

After 10 hours, the oxygen consumption rate was kept at about 70 mmol/(L·h), the electrical conductivity of the fermentation broth was controlled to be 12 ms/cm, pH was controlled to about 6.0, and the fermentation was continued. The fermentation at this time was within the early stage of the synthesis and accumulation stage of coenzyme Q10.

After 20 hours, the increase in fermentation potency tended to reach a balance at this time, and the fermentation entered the later period of the synthesis and accumulation stage of coenzyme Q10. The air intake flow rate per unit volume of the fermentation broth in the fermentation tank was controlled to be 6.0 vvm, the input power of stirring per unit volume was controlled to be 0.3 kw/m³, the tank pressure was 0.1 MPa, the pH value was adjusted to about 4.0 over about 2 h by continuously adding phosphoric acid, the electrical conductivity of the fermentation broth was controlled to be 12 ms/cm, and the fermentation was continued. The ORP value of the fermentation broth was maintained between 100 to 200 my after reaching a steady state.

After 15 hours, the fermentation was terminated. Partial fermentation broth was taken, extracted and tested under an inert gas atmosphere. The potency was 3533 mg/L, oxidized coenzyme Q10: reduced coenzyme Q10 was 99.3:0.7, and the biomass was 123 g/kg.

### Comparative Example 1 Construction of a Recombinant Microorganism without being Subjected to Osmoregulated Promoter proPB Replacement

The recombinant vector pBBR1MCS-2-irrE constructed in Example 1 was not subjected to the osmoregulated promoter proPB replacement. Said recombinant vector pBBR1MCS-2-irrE was transformed into Escherichia coli S17-1 competent cells with reference to Example 3 and cultured on an LB medium containing kanamycin for 24 h, so as to obtain a recombinant Escherichia coli. The recombinant Escherichia coli was picked and the plasmid was extracted. PCR verification was conducted by using primers irrE-F and irrE-R, and a fragment of approximately 1.0 kb was obtained, indicating that the gene encoding the global regulatory protein irrE had been successfully introduced into Escherichia coli S17-1.

The recombinant vector pBBR1MCS-2-irrE in the obtained recombinant Escherichia coli S17-1 was introduced into Rhodobacter sphaeroides by conjugative transfer, and was cultured using a plate medium containing nalidixic acid and kanamycin, so as to obtain the recombinant Rhodobacter sphaeroides RSP-CE. After PCR verification was conducted by using primers irrE-F and irrE-R, a fragment of approximately 1.0 kb was obtained, indicating that the gene encoding the global regulatory protein irrE had been successfully introduced into Rhodobacter sphaeroides RSP-CE.

### Comparative Example 2 Comparison of Recombinant Microorganisms Used in Coenzyme Q10 Fermentation

The original strain of Rhodobacter sphaeroides, the recombinant Rhodobacter sphaeroides RSP-BE and the recombinant Rhodobacter sphaeroides RSP-CE were subjected to fermentation with reference to the fermentation method in Example 4, and the results of fermentation were as follows.

| | Potency of | |
|---|---|---|
| Type of strain | coenzyme Q10 | Biomass |
| Original | 2975 mg/L | 110 g/kg |
| strain | | |
| RSP-CE | 3276 mg/L | 119 g/kg |
| RSP-BE | 3510 mg/L | 123 g/kg |

### Comparative Example 3 Comparison of Recombinant Microorganisms Used in Oxidized Coenzyme Q10 Fermentation

The original strain of Rhodobacter sphaeroides, the recombinant Rhodobacter sphaeroides RSP-BE and the recombinant Rhodobacter sphaeroides RSP-CE were subjected to fermentation with reference to the fermentation method in Example 5, and the results of fermentation were as follows.

| | Potency of | | |
|---|---|---|---|
| | oxidized | Oxidized coenzyme | |
| Type of | coenzyme | Q10:Reduced | |
| strain | Q10 | coenzyme Q10 | Biomass |
| Original | 2780 mg/L | 96.3:3.7 | 100 g/kg |
| strain | | | |
| RSP-CE | 3113 mg/L | 98.5:1.5 | 113 g/kg |
| RSP-BE | 3436 mg/L | 99.6:0.4 | 120 g/kg |

The results of Comparative Example 3 showed that the potency of oxidized coenzyme Q10 obtained by the fermentation of the original strain of Rhodobacter sphaeroides and the ratio of said oxidized coenzyme Q10 to reduced coenzyme Q10 were low due to the adverse effects of high redox potential on the bacteria during the fermentation production process. Since the gene encoding the global regulatory protein irrE played a central regulatory role in the pathways of the repair of DNA damage and the protection from the response of radiation stress, introducing the gene encoding the global regulatory protein irrE exogenously made it possible to enhance the tolerance of the microorganism against harsh environments, including the tolerance against a variety of stresses such as osmotic pressure, oxidation, radiation and heat, which was beneficial to the growth and metabolic activity of the strain as well as the increase of the biomass, and increased the potency and the relative proportion of oxidized coenzyme Q10 to some extent. However, due to the lack of the osmoregulated promoter proPB replacement, the potency detected in the fermentation broth of the recombinant Rhodobacter sphaeroides strain RSP-CE was lower, as compared with the recombinant Rhodobacter sphaeroides strain RSP-BE. It could be seen that the osmoregulated promoter proPB was able to effectively regulate the expression of irrE according to the change of the conditions in the actual fermentation environment, thus enhancing the tolerance of the coenzyme Q10-producing strain against stresses of different intensities.

### INDUSTRIAL AVAILABILITY

Since the recombinant microorganism provided by the present disclosure used for the production of coenzyme Q10 by fermentation method comprises the gene encoding the global regulatory protein irrE, it is possible to enhance the tolerance of the microorganism against a variety of stresses such as osmotic pressure, oxidation, radiation and heat, thus not only prolonging the logarithmic growth phase of the strain and promoting further accumulation of biomass, but also maintaining vigorous growth and metabolic activity of the strain during the fermentation process, thereby increasing the yield of coenzyme Q10, especially the yield of oxidized coenzyme Q10. Therefore, the recombinant microorganism constructed by this gene is able to increase the potency of coenzyme Q10 advantageously, in particular, significantly increase the content of oxidized coenzyme Q10. Accordingly, the recombinant microorganism constructed by the method of the present disclosure has broad application prospect in the industrial production of coenzyme Q10.

While the invention has been described in connection with what is presently considered to be the most practical and preferred embodiment, it is to be understood that the invention is not to be limited to the disclosed embodiments but, on the contrary, is intended to cover various modifications and equivalent arrangements included within the spirit and scope of the appended claims, which scope is to be accorded the broadest interpretation so as to encompass all such modifications and equivalent structures as is permitted under the law.

1. A method for generating a recombinant microorganism, wherein the method comprises the following steps:
step a. cloning a gene encoding a global regulatory protein irrE from a parent strain comprising the gene encoding the global regulatory protein irrE;
step b. ligating the gene encoding the global regulatory protein irrE to a vector, and constructing a recombinant vector comprising the gene encoding the global regulatory protein irrE; and
step c. introducing the recombinant vector into a host cell so as to obtain the recombinant microorganism.

2. The method for generating the recombinant microorganism according to claim 1, wherein said step b includes knocking out a promoter in the recombinant vector in which said promoter controls the expression of the gene encoding the global regulatory protein irrE and then inserting other different promoter(s) by promoter replacement, so as to further regulate the expression of the gene encoding the global regulatory protein irrE.

3. The method for generating the recombinant microorganism according to claim 1, wherein the promoter inserted in said step b is an inducible promoter, preferably an osmoregulated promoter proPB, and
wherein the osmoregulated promoter proPB is obtained from a polynucleotide molecule or a polynucleotide sequence comprising a partial nucleotide sequence of at least 70 consecutive nucleotides of SEQ ID NO: 2, preferably comprising at least 100 consecutive nucleotides of SEQ ID NO: 2, more preferably comprising at least 150 consecutive nucleotides of SEQ ID NO: 2, and most preferably comprising the whole nucleotide sequence of SEQ ID NO: 2, the polynucleotide sequence has at least 60% homology, preferably at least 80% homology and more preferably at least 90% homology with SEQ ID NO: 2, and preferably, the osmoregulated promoter proPB is a nucleotide sequence set forth in SEQ ID NO: 2.

4. The method for generating the recombinant microorganism according to claim 3, wherein the osmoregulated promoter proPB is isolated from a bacterium.

5. The method for generating the recombinant microorganism according to claim 1, wherein the vector in said step b is selected from pBR322, derivatives of pBR322, pACYC177, pACYC184, derivatives of pACYC184, RK2, pBBR1MCS-2 and a cosmid vector and its derivatives.

6. The method for generating the recombinant microorganism according to claim 1, wherein said step a includes designing a primer according to a DNA sequence set forth in SEQ ID NO: 1, using a genomic DNA extracted from the parent strain as a template, and synthesizing the gene encoding the global regulatory protein irrE by a PCR method.

7. The method for generating the recombinant microorganism according to claim 1, wherein the gene encoding the global regulatory protein irrE in said step a is obtained from a polynucleotide molecule or a polynucleotide sequence comprising a partial nucleotide sequence of at least 100 consecutive nucleotides of SEQ ID NO: 1, preferably comprising at least 300 consecutive nucleotides of SEQ ID NO: 1, the polynucleotide sequence has at least 60% homology, the gene encoding the global regulatory protein irrE is a nucleotide sequence set forth in SEQ ID NO: 1.

8. The method for generating the recombinant microorganism according to claim 1, wherein the parent strain is a bacterium from genus Deinococcus.

9. The method for generating the recombinant microorganism according to claim 1, wherein a way of introducing in said step c is selected from transformation, transduction, conjugative transfer and electroporation, the host cell is selected from bacteria or fungi.

10. A recombinant vector, wherein the recombinant vector comprises the gene encoding the global regulatory protein irrE of claim 7, and the osmoregulated promoter proPB of claim 3.

11. A recombinant microorganism, wherein the recombinant microorganism comprises the gene encoding the global regulatory protein irrE of claim 7, and the osmoregulated promoter proPB of claim 3.

12. A method for producing coenzyme Q10, wherein the method comprises generating a recombinant microorganism by using the method of claim 1 and producing coenzyme Q10 by using the recombinant microorganism.

13. A method for producing oxidized coenzyme Q10, wherein the method comprises generating a recombinant microorganism by using the method of claim 1 and producing oxidized coenzyme Q10 by using the recombinant microorganism.

14. The method for generating the recombinant microorganism according to claim 2, wherein the promoter inserted in said step b is an inducible promoter, preferably an osmoregulated promoter proPB,
wherein the osmoregulated promoter proPB is obtained from a polynucleotide molecule or a polynucleotide sequence comprising a partial nucleotide sequence of at least 70 consecutive nucleotides of SEQ ID NO: 2, preferably comprising at least 100 consecutive nucleotides of SEQ ID NO: 2, more preferably comprising at least 150 consecutive nucleotides of SEQ ID NO: 2, and most preferably comprising the whole nucleotide sequence of SEQ ID NO: 2, the polynucleotide sequence has at least 60% homology, preferably at least 80% homology and more preferably at least 90% homology with SEQ ID NO: 2, and preferably, the osmoregulated promoter proPB is a nucleotide sequence set forth in SEQ ID NO: 2, and
wherein the osmoregulated promoter proPB is isolated from a bacterium from genus Escherichia.

15. The method for generating the recombinant microorganism according to claim 14, wherein the vector in said step b is selected from pBR322, derivatives of pBR322, pACYC177, pACYC184, derivatives of pACYC184, RK2, pBBR1MCS-2 and a cosmid vector and its derivatives.

16. The method for generating the recombinant microorganism according to claim 15 wherein said step a includes designing a primer according to a DNA sequence set forth in SEQ ID NO: 1, using a genomic DNA extracted from the parent strain as a template, and synthesizing the gene encoding the global regulatory protein irrE by a PCR method, wherein the gene encoding the global regulatory protein irrE in said step a is obtained from a polynucleotide molecule or a polynucleotide sequence comprising a partial nucleotide sequence of at least 100 consecutive nucleotides of SEQ ID NO: 1, preferably comprising at least 600 consecutive nucleotides of SEQ ID NO: 1, the polynucleotide sequence has at least 80% homology, the gene encoding the global regulatory protein irrE is a nucleotide sequence set forth in SEQ ID NO: 1.

17. The method for generating the recombinant microorganism according to claim 14, wherein the parent strain is a bacterium selected from the group consisting of Deinococcus radiodurans, Deinococcus deserti, Deinococcus gobiensis and Deinococcus proteolyticus, and mixtures thereof.

18. The method for generating the recombinant microorganism according to claim 15, wherein a way of introducing in said step c is selected from transformation, transduction, conjugative transfer and electroporation, the host cell is selected from bacteria from genus Rhodobacter and wherein step c includes transforming the recombinant vector obtained in said step b to an Escherichia coli S17-1 competent cell and then introducing the recombinant vector into the host cell by conjugative transfer, so as to obtain a genetically stable recombinant microorganism.

19. A method for producing coenzyme Q10, wherein the method comprises generating a recombinant microorganism by using the method of claim 14 and producing coenzyme Q10 by using the recombinant microorganism.

20. A method for producing oxidized coenzyme Q10, wherein the method comprises generating a recombinant microorganism by using the method of claim 14 and producing oxidized coenzyme Q10 by using the recombinant microorganism.


SEQUENCE LISTING (8 sequences)

SEQ ID NO: 1 - DNA, 987 bp, Artificial Sequence
  Synthetic
gtgcccagtgccaacgtcagccccccttgcccctctggggtaaggggcggggggatggggccaaaagctaaagctgaagcctccaagccccacccccaaatccctgttaagctcccattcgtgaccgcccccgacgccctcgccgccgccaaagccaggatgcgcgacctggcggcggcctacgtggcggccctgcccggacgcgacacccacagcctgatggcgggggtgcccggcgtagacctcaaattcatgccgctcggctggcgcgacggggcgttcgaccccgagcacaacgtcatcctcatcaactcggcggcccgccccgaacgccagcgcttcaccctcgcccacgaaatcgggcacgcgattttactcggcgacgacgacctgctctccgacatccacgacgcctacgagggcgagcggctcgaacaggtcatcgaaacgctgtgcaacgtggcggcggcggcgattttgatgcccgaacccgtcatcgcggaaatgctggaacgcttcggccccaccgggcgcgccctcgccgaactcgccaagcgggccgaagtcagcgcgtcgtcggcgctctacgccctgaccgagcagaccccggtgcccgtcatctacgcggtctgtgcgccgggcaagcctccgcgtgagcaggccgcaagcgacgaggacgctggcccaagcacagaaaaagtcctgacggtccgcgccagcagctcgacgcggggcgtcaagtacaccctggcgagcggcacgccggtacccgccgaccacccggcggcgcttgccctcgccacgggcatggaagtgcgcgaggaaagctacgtgccctttcgctcgggccggaaaatgaaggcggaggtggacgcctacccgtcgcgcggcatcgtggccgtcagtttcgagttcgaccccgcccgcctgggccgcaaggacagcgagcaggccgaccgggacgagccgcaggacgctgcacagtga

SEQ ID NO: 2 - DNA, 210 bp, Artificial Sequence
  Synthetic
tgtgtgaagttgatcacaaatttaaacactggtagggtaaaaaggtcattaactgcccaattcaggcgtcaactggtttgattgtacattccttaaccggagggtgtaagcaaacccgctacgcttgttacagagattgcatcctgcaattcccgctccccttttgcggccgtcgcgctgatttttctggcgtttgcggaaatgggccaa

SEQ ID NO: 3 - DNA, 38 bp, Artificial Sequence
  Synthetic
ccggaattcgtgcccagtgccaacgtcagccccccttg

SEQ ID NO: 4 - DNA, 36 bp, Artificial Sequence
  Synthetic
cgcggatcctcactgtgcagcgtcctgcggctcgtc

SEQ ID NO: 5 - DNA, 42 bp, Artificial Sequence
  Synthetic
gcctggggtgcctaatgagtgagctaactcacattaattgcg

SEQ ID NO: 6 - DNA, 47 bp, Artificial Sequence
  Synthetic
ctcattaggcaccccaggctgtggaattgtgagcggataacaatttc

SEQ ID NO: 7 - DNA, 34 bp, Artificial Sequence
  Synthetic
ccgctcgagcatgtgtgaagttgatcacaaattt

SEQ ID NO: 8 - DNA, 32 bp, Artificial Sequence
  Synthetic
cccaagcttgagttggcccatttccgcaaacg